(19) United States
(12) Patent Application Publication
(10) Pub. No.: US 2007/0178576 A1
(43) Pub. Date: Aug. 2, 2007

(54) Purification of high-molecular compounds by means of affinity membrane chromatography

(75) Inventors: Wolfgang Demmer, Gottingen (DE); Matthias Grabosch, Bovenden (DE); Oscar-Werner Reif, Hannover (DE); Wolfgan Nagel, Munchen (DE); Mariel Donzeau, Munchen (DE); Peter Reichelt, Munchen (DE)

Correspondence Address: MCDONNELL BOEHNEN HULBERT & BERGHOFF LLP, 300 S. WACKER DRIVE, 32ND FLOOR, CHICAGO, IL, 60606, US

(73) Assignees: Sartorius AG, Gottingen (DE); Apanovis Biotechnologie GmbH, Martinsried (DE)

(21) Appl. No.: 10/587,064

(22) PCT Filed: Jan. 27, 2005
(86) PCT No.: PCT/EP05/00810

§ 371 Date: Aug. 8, 2006

(30) Foreign Application Priority Data
Jan. 27, 2004 (DE) 10 2004 004 043.5

(51) Int. Cl. C12N 7/02; C07K 14/47; C12N 5/06

(52) U.S. Cl. 435/239; 435/325; 530/414

(57) ABSTRACT

The invention relates to a method for purifying and/or isolating high-molecular compounds, such as high-molecular proteins or protein-type compounds, contained in a solution or a suspension, by means of affinity chromatography using membranes containing metal ions.

The present invention relates to a method for purifying and/or isolating high-molecular compounds, such as high-molecular proteins or protein-type compounds, contained in a solution or a suspension, by means of affinity chromatography using metal ions containing membranes.

Chromatographic methods play an important role in the purification and isolation of proteins or protein-type compounds, especially when they are used as vaccine. Affinity chromatographic methods for purifying recombinant bacteriophages are described in the prior art, for example, which methods, however, are disadvantageous among others in that they exhibit a very low binding capacity of the chromatography material used (see Bass et al., 1990; McCafferty et al., 1991; U.S. Pat. No. 5,750,373; U.S. Pat. No. 5,688,666; WO 92/09690).

Thus, in the production of filamentous bacteriophages, which carry or express recombinant proteins on their surface, wherein such recombinant proteins can, for example, be antigens for use as therapeutic vaccines against cancer or autoimmune diseases, or antigens for the use of prophylactic vaccines against infectious diseases, in most cases a mixture of bacteriophages is obtained, said bacteriophages carrying many, few or no recombinant proteins, wherein the latter case relates to wild type bacteriophages. Varying levels of expression of the antigen are achieved, depending upon the antigen used. This means that the proportion of phages that carry antigens can vary, depending upon the antigen used. Thus, in contrast to other viral particles, which usually constitute the antigen themselves, a special problem arises in the production of recombinant bacteriophages, namely the separation of the wild type bacteriophages from the desired bacteriophages, which carry the recombinant protein, an antigen, for example, on the surface. One of the factors in the use of recombinant phage vaccines is the density of the recombinant antigen expressed on the surface of the phage, because the actual goal of immunization is to induce a specific immune response to the expressed recombinant antigen. In this connection, the wild type bacteriophages can play an interfering role, because the goal is not the immune response to the bacteriophages, but rather an immune response to the “foreign” antigens that are expressed on the phage. As a result of high levels of contamination with wild type bacteriophages, the relationship between the immune reaction against the antigen and the immune reaction against the phages worsens. For this reason, it is desirable to purify the bacteriophages that have the desired antigen on their surface and to separate the unwanted wild type bacteriophages.

In addition, it is desirable, especially in the case of recombinant vaccines, to separate contaminating molecules of all types, especially proteins and endotoxins of the host organism used to produce the recombinant vaccines, such as E. coli or mammalian cells, and potentially contaminating proteins and low-molecular compounds, such as antibiotics, cytokines, etc., from the culture medium of bacteria or mammalian cells, for example, from the recombinant vaccines, so as to achieve the greatest possible purity.

Another important requirement for the purification process for genetically-engineered proteins, (poly)peptides or recombinant bacteriophages that can be used as vaccines is manufacturability on a commercial scale, so that amounts of recombinant bacteriophages, for example, can be produced that are sufficient for use in human beings.

The structural characteristics of particulate substances, such as multimer protein complexes, multi-enzyme complexes, recombinant viruses, VLPs and bacteriophages, represent a problem for conventional, particulate-based purification mediums, wherein such substances cannot or can only be inadequately purified by such particulate-based mediums. This is illustrated by the following example of filamentous bacteriophages.

Fd-phages have an unusual shape (for example, the fd-phage has a diameter of 6 nm and a length of up to 900 nm) and a molecular weight of about 15×10⁶ Dalton, which poses special challenges with regard to purification. A method for producing phage vaccines against tumors is described in PCT/EP99/03380. Although these vaccines are already being used successfully, it would be desirable to further simplify the manufacturing process for the large-scale use described therein.

Therefore, the problem underlying the present invention is to provide a method that permits cost-efficient and rapid concentration, purification and/or isolation, especially on a commercial scale, of high-molecular compounds, such as high-molecular proteins or protein-like compounds, such as filamentous bacteriophages carrying antigens that do not occur naturally on the surface.

This problem is solved by providing the embodiments characterized in the claims.

In particular, a method is provided for purifying and/or isolating high-molecular compounds contained in a solution or suspension, such as high-molecular proteins or high-molecular protein-like compounds, said method comprising the steps:
(a) Application of the solution or suspension onto a metal ions containing membrane, and
(b) affinity chromatographic separation of the high-molecular compounds by binding them to the metal ions containing membrane, wherein the high-molecular compounds have the capacity to form metal chelate and preferably have a molecular weight greater than 1×10⁶ Da, more preferably greater than 2×10⁶ and especially preferably greater than 5×10⁶ Da.

Because of their size and/or shape, the high-molecular compounds, such as proteins or protein-like compounds, essentially cannot be purified and/or isolated through conventional chromatography material, such as modified agarose, e.g. Sepharose®. For example, viruses can have an icosahedral, helical or complex form and can be coated or non-coated, with viruses having, for example, a size of about 10 nm to about 100 nm. In special cases, viruses are rod-shaped and have a length of several hundred nanometers. For example, phage M13 has a length of about 900 nm. In particular, in gel-chromatographic separation and/or isolation the high-molecular compounds cannot substantially penetrate into the hetero-porous, swollen network of pearls or “beads” (such as Sepharose®) that are conventionally used as a stationary phase in gel chromatography.

The term “metal ion(s)” is not subject to any particular limitation, to the extent that the metal ions used have a specific affinity for the proteins or protein-like compounds to be purified and/or isolated. Preferred metal ions are selected from the group consisting of Cu²⁺, Ni²⁺, Zn²⁺, Co³⁺, Fe³⁺, Mn²⁺ and Ca²⁺, and mixtures of at least two of these metal ions. The metal ion Cu²⁺ is especially preferred.

The matrix material of the membrane used according to the invention is not subject to any special limitation and is preferably selected from the group consisting of agaroses, modified agaroses, modified dextranes, polystyrenes, polyethers, polyacrylamides, polyamides, e.g., nylon, cellulose, modified celluloses, such as cross-linked celluloses, nitrocelluloses, cellulose acetates, silicates and poly(meth)acrylates, polytetrafluoroethylene, polyesters, polyvinyl chlorides, polyvinylidene fluoride, polypropylene, polysulfones and polyethersulfones. The pore size of the membrane used according to the invention is preferably in the range of 0.01 to 12 μm, preferably in the range of 0.45 to 7 μm, and especially preferably the range of 3 to 5 μm.

The term “high-molecular compounds” encompasses high-molecular proteins and high-molecular protein-like compounds, as well as biopolymers, lipids, micelles and liposomes.

The term “protein-like compound(s)” encompasses (poly)peptides and derivatives thereof, derivatized proteins, recombinant proteins and (poly)peptides, di-, tri-, tetra- to multimers of peptides, polypeptides or proteins, (multi)-protein complexes, cell organelles, fusion proteins, viruses and parts thereof, such as shell proteins, recombinant viruses and parts thereof, recombinant bacteriophages, such as recombinant filamentous bacteriophages, and parts thereof, which carry on their surface antigens that do not occur naturally. In a preferred embodiment of the method of the invention, recombinant, filamentous bacteriophages are purified and/or isolated as protein-like compounds.

The term “filamentous bacteriophage” comprises, within the meaning of this invention, all phages that tend to have a helical rather than an icosaedric symmetry. The filamentous bacteriophage can be a class I phage, such as fd-, M13-, f1-, lf1-, lke-, ZJ/Z- or Ff-phage, or a class II phage, such as Xf-, Pfl1- or Pf3-phage. In an especially preferred embodiment of the method according to the invention, the protein-like compound is the filamentous bacteriophage M13.

The term “antigens that do not occur naturally” refers, in the context of this invention, to antigens, such as proteins, that do not occur in the capsids of the wild type forms of filamentous bacteriophages. These are antigens that can be recombinantly expressed by the phages and built into the capsid, or that can be bonded chemically, for example, to the capsid.

In a further preferred embodiment of the method of the invention, the recombinantly expressed protein on the bacteriophage is a fusion protein with protein III and/or protein VIII of the bacteriophage.

The antigen must be a fusion protein with a phage protein if it is to be recombinantly expressed on the phage. Otherwise it cannot be incorporated. In the case of protein III, a portion of this protein is also sufficient, because said protein is located on the head of the phage and is only connected to the phage at one end. In the case of protein VIII, the entire protein is necessary, because incorporation into the phages cannot otherwise occur (p VIII is relatively and small and forms the shell of the phage). The advantage of incorporating the antigen through recombinant expression as a fusion protein is that the presentation is oriented toward the phage surface and/or is precisely defined.

The expression “purification of high-molecular compounds, such as proteins or protein-like compounds, such as recombinant, filamentous bacteriophages” refers, within the meaning of this invention, to the fact that at least 97%, preferably at least 98%, especially preferably at least 99% and most preferably at least 99.8% of the proteins or protein-like compounds to be purified by the method are present.

In this invention, the expression “isolation of high-molecular compounds, such as proteins or protein-like compounds, such as recombinant, filamentous bacteriophages” refers to the fact that all high-molecular compounds purified by the method are essentially pure. The isolated high-molecular compounds preferably no longer contain any contaminating proteins, such as, in the case of isolated bacteriophages, any contaminating E. coli proteins, and/or any culture medium components.

In a preferred embodiment of the method of the invention, recombinant, antigen-carrying bacteriophages are purified and/or isolated, wherein it is surprisingly possible to purify and/or isolate at least 1×10¹³ antigen-carrying bacteriophages per 50 to 100 cm² membrane surface from a mixture that contains both wild type bacteriophages and antigen-carrying bacteriophages.

Because of the surprisingly large amount of purified material per surface unit of the membrane, combined with the high degree of purity exhibited by the high-molecular compounds, such as proteins or protein-like compounds, that are purified and/or isolated according to the method of the invention, it is possible to produce and use said compounds as vaccines on a commercial scale. This is especially applicable to the recombinant bacteriophages. The unexpected advantages of the invention are characterized, in particular, by a substantially higher level of effectiveness, in animal experiments, when compared with the non-purified bacteriophages. Testing of the effectiveness of a vaccine is known to the person skilled in the art and is thus prior art.

The principle of metal ion affinity chromatography is based on metal chelate formation and complex formation, respectively, between metal ions, such as Cu²⁺, Ni²⁺, Zn²⁺, Co³⁺, and the protein to be purified, which preferably has a sequence of 5 to 6 histidine radicals (“His-Tag”) or a plurality of such units in sequence. The metal ion can be linked to the membrane matrix through an additional complex forming agent. This is based on the principle that many metals, such as copper and zinc, and their ions can form coordination complexes with the amino acids histidine, cysteine and tryptophan through electron donor groups of the side chains of the amino acids. To be able to utilize this effect in chromatography, these metal ions must be immobilized on an inert matrix. This can be achieved by applying a chelate-forming group onto the matrix. Of special importance, with regard to the use of such groups, is that they are fixed on the matrix and have a high affinity for the substance to be purified. The most common chelate-forming ligand in this type of chromatography is imino-diacetic acid (IDA). It forms a chelate (multiple coordination sites) with metals. The most common metals are copper and zinc, although others, such as cobalt, nickel, iron, lanthanum, manganese and calcium, have been described. The His-Tag can be located in the protein at the N-terminus, at the C-terminus or internally. The special aspect of the purification of recombinant bacteriophages according to the invention is that it does not apply to a single protein, but rather to a phage particle that consists of thousands of proteins or phage particle fragments that consist of smaller multimers and have a highly unusual shape.

A “Tag,” within the meaning of the invention, is a binding partner that forms a fusion protein with the protein or protein-like compound to be purified, wherein the specific interaction then takes place between the Tag and a metal ion specific to the Tag. In an especially preferred embodiment of the method of the invention, the Tag is selected from the group consisting of His-Tag, Flag-Tag and Myc-Tag.

In addition, naturally occurring protein motives with metal chelate characteristics, such as so-called zinc finger motives of transcription factors, are to be understood as tags within the meaning of the invention, because they are capable of performing a special interaction with metal ions.

The shape of the metal ion containing membrane used in accordance with the invention is not subject to any particular limitation and can be arranged in a casing, such as a plastic casing or a chromatography column. A planar form of the metal ions containing membrane is preferred, wherein several layers of the metal ion containing membrane can be arranged in a package for a chromatography column on top of one another.

In the method according to the invention, a mixture containing the high-molecular compounds to be purified and/or isolated can be subjected to ion exchange chromatography to remove impurities prior to step (a). The ion exchange chromatography is preferably performed using an ion exchanger membrane. The ion exchanger membrane preferably comprising a matrix material selected from the group consisting of agaroses, modified agaroses, modified dextranes, polystyrenes, polyethers, polyacrylamides, polyamides, e.g., nylon, cellulose, modified celluloses, such as cross-linked celluloses, nitrocelluloses, cellulose acetates, silicates and poly(meth)acrylates, polytetrafluoroethylene, polyesters, polyvinyl chlorides, polyvinylidene fluoride, polypropylene, polysulfones and polyethersulfones. The ion exchanger membrane preferably has a pore size in the range of 0.01 to 12 μm, preferably in the range of 0.45 to 7 μm, especially preferably in the range of 3 to 5 μm. The functional groups of the ion exchanger membrane are not subject to any particular limitation and are, for example, adjusted according to the protein to be purified or the protein-like compound to be purified. Preferred examples of functional groups are DEAE, DEA, CM, QA, TMA, S, SP and phosphate groups.

Examples of impurities that can be removed through ion exchange chromatography, e.g., by means of binding to the ion exchanger membrane, are, in particular, endotoxins, which originate for example from the host organism, e.g. E. coli, in the case of recombinant production of protein or protein-like compounds such as recombinant filamentous bacteriophages.

It is also possible to optimize the method in such a way that, prior to the affinity membrane chromatography and/or prior to the ion exchange (membrane) chromatography, as many impurities as possible are further reduced using, for example, the precipitation steps known in the prior art or the filtration steps known in the prior art, thereby keeping the degree of contamination as low as possible. This reduces the probability of adverse side effects during the use as a biologically active substance, especially as a vaccine.

In a preferred embodiment, the mixture, which contains, for example, the protein to be purified or the protein-like compound to be purified, such as recombinant filamentous bacteriophages, is subjected to filtration using a commercially available filtration system, such as the cross-flow microfiltration system by the Sartorius Company, prior to the affinity membrane chromatography and/or the ion exchange (membrane) chromatography. In this connection, a filter cassette, for example, with a Hydrosart® membrane having a nominal pore size of 0.45 μm or 0.2 μm, is used in a suitable clamping device. The operation of such systems is known to the person skilled in the art. Additional impurities, such as the host organisms or parts thereof, can be removed through said filtration. The filtrate obtained in this manner can then be used in ion exchange chromatography and/or affinity membrane chromatography, optionally after suitable conventional processing.

The method according to the invention can be performed either as a “batch” process or continuously.

In a further preferred embodiment, the method according to the invention comprises the further formulation of the purified and/or isolated high-molecular compound, e.g. a protein or a protein-like compound, as a vaccine. For example, phages are dialyzed against PBC following purification, after which the protein concentration, the PFU, the endotoxin concentration and the antigen quantity is determined. The endotoxin concentration and the antigen quantity are preferably determined using the LAL test and an immune dot blot, respectively. The concentration is adjusted by diluting to the desired level.

In a further, especially preferred embodiment, the method is performed under GMP conditions, wherein GMP refers to “good manufacturing practice” and is known to the person skilled in the art, and thus constitutes the prior art.

Another object of the present invention relates to the use of the high-molecular compounds produced in accordance with the invention, such as proteins or protein-like compounds, preferably recombinant filamentous bacteriophages, as biologically and/or pharmacologically active components in a pharmaceutical compositions, such as a vaccine, which optionally includes a pharmaceutically acceptable carrier and/or diluent. Examples of suitable carriers or diluents are known to the person skilled in the art and comprise, for example, phosphate-buffered saline solutions, water, emulsions, such as oil/water emulsions, various types of wetting agents or detergents, sterile solutions, etc. Compositions that comprise such carriers and/or diluents can be produced by means of known, conventional methods.

The pharmaceutical compositions can be administered to an individual in suitable doses. Administration can be oral or parenteral, e.g., using intravenous, intraperitoneal, subcutaneous, perinodal, intramuscular, topical, intradermal, intranasal or intrabronchial methods, or through a catheter in an artery. The level of the dose is determined by the treating physician and depends substantially on the clinical factors. These factors are known in the art and comprise, for example, height and/or weight, body surface, age, gender and the patient's general state of health, the specific composition to be administered, the duration of treatment, the form of administration and, if applicable, concomitant treatment with another drug. A typical dose can range, for example, between 0.001 and 5,000 μg, although doses below or above this illustrative range are conceivable, especially when the above-mentioned factors are taken into account. In general, the dose should range between 1 μg and 10 mg units per day when the composition according to the invention is administered regularly. If the composition is administered intravenously, which is not preferably recommended, so as to minimize the risk of an anaphylactic reaction, the dose should preferably fall within a range of about 1 μg to about 10 mg units per kg of body weight, per minute.

The pharmaceutical composition can be administered locally or systemically. Preparations for parenteral administration comprise sterile aqueous or non-aqueous solutions, suspensions and emulsions. Examples of non-aqueous solvents are propylene glycol, polyethylene glycol, vegetable oils, such as olive oil, and organic ester compounds, such as ethyl oleate, that are suitable for injection. Aqueous carriers comprise water, alcoholic aqueous solutions, emulsions, suspensions, saline solutions and buffered mediums. Parenteral carriers comprise sodium chloride solutions, ringer dextrose, dextrose, sodium chloride, ringer lactate and combined oils. Intravenous carriers comprise, for example, liquid, nutrient and electrolyte supplements (such as those based on ringer dextrose). The composition according to the invention can also comprise preservatives and other additives, such as antimicrobial compounds, antioxidants, complex-forming agents and inert gases. In addition, depending on the intended use, compounds such as interleukins, growth factors, differentiation factors, interferons, chemotactic proteins or a non-specific immune-modulating agent can be contained.

The figures show:

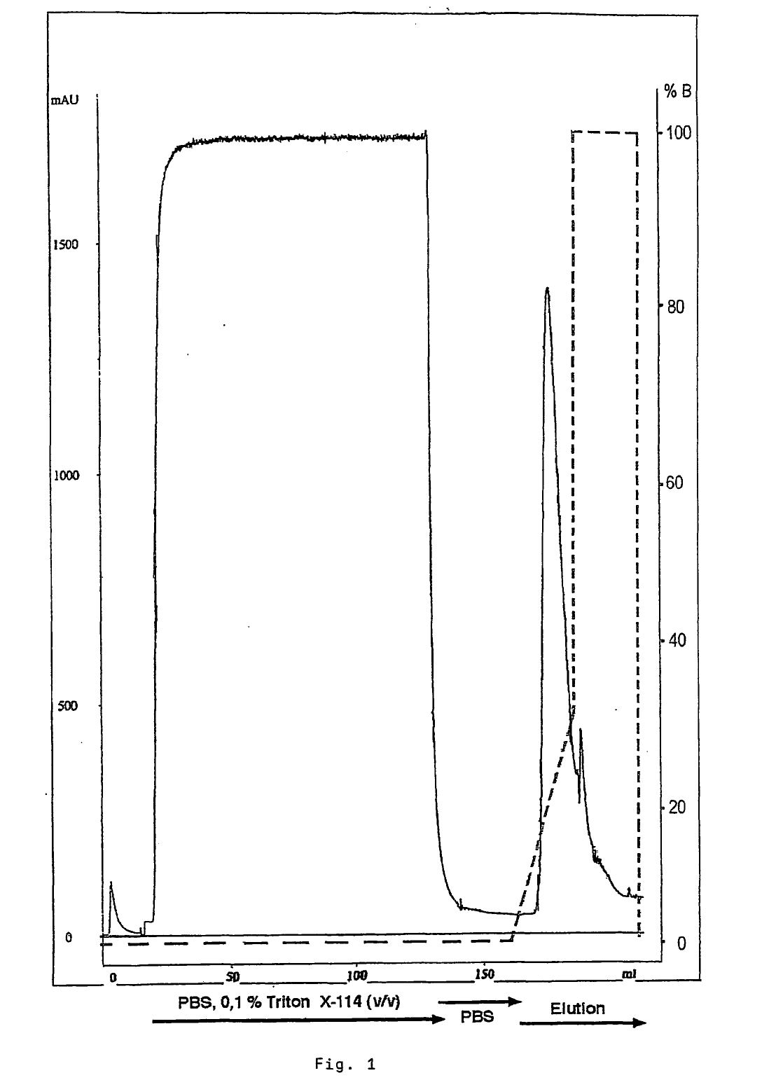

FIG. 1 shows a graphic depiction of the washing and elution steps of a ion exchange chromatography. The first signal contains the main fraction of endotoxins and the second signal essentially contains the phages. The dashed line represents the NaCl gradient.

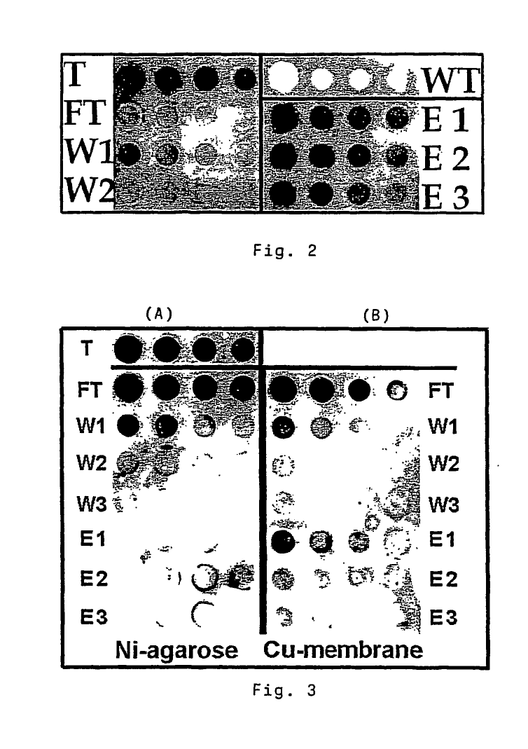

FIG. 2 is a photographic image of a dot blot relating to the purification of recombinant M13/fd phages with His-Tag/g8 fusion proteins, wherein monoclonal peroxidase-conjugated/anti-polyhistidine antibodies are used for immunological detection. Dilution series from left to right: 1/100, 1/200, 1/400, 1/800; T=total; FT=flow; W1, W2, W3=respective washing steps; E1, E2, E3=respective elution steps; WT=wild type phage (see example 4).

FIG. 3 is a photographic image of a dot blot relating to the purification of recombinant M13/fd phages with His-Tag/g8 fusion proteins, wherein monoclonal peroxidase-conjugated/anti-polyhistidine antibodies are used for immunological detection. Dilution series from left to right: 1/100, 1/200, 1/400, 1/800; T=total; FT=flow; W1, W2, W3=respective washing steps; E1, E2, E3=respective elution steps; WT=wild type phage (see example 4). (A) Commercially available Ni²⁺⁻ agarose beads are used as chromatography material. (B) A Cu²⁺-containing membrane according to the invention is used as chromatography material.

The following examples illustrate the invention without limiting the scope of protection.

### EXAMPLES

### Example 1

Cross-flow filtration of bacteriophage M13 -containing solutions and suspensions, respectively.

2 l of a bacteriophage M13 culture are subjected to cross-flow filtration using a Hydrosart membrane (pore size 0.4 μm) by Sartorius.

The results show that a phage titer of 8×10⁴ phages/l can be obtained using the single-step method of cross-flow filtration, which corresponds approximately to the result achieved by isolating the phages using the two-step “centrifugation and PEG/NaCl precipitation” known in the prior art.

In addition, contamination with bacterial cells can be substantially prevented by using cross-flow filtration. Cross-flow filtration thus presents a simple and quick method for separating phages from bacteria cells that produces a yield that is at least as favorable as that achieved with the “centrifugation and PEG/NaCl precipitation” method known in the prior art. The endotoxin content of the phages obtained by cross-flow filtration corresponds approximately to that achieved with the “centrifugation and PEG/NaCl precipitation” method.

### Example 2

Ion exchange chromatography of bacteriophage M13-containing solutions and suspensions, respectively.

2 ml of a solution containing 7 mg of M13 phages, which is derived from example 1, are subjected to ion exchange chromatography at 4° C., using a Q75 ion exchange membrane obtainable from Sartorius. The binding of the phages to the membranes is accomplished with a binding buffer (PBS, pH 7). For elution of the endotoxins, which are also bound to the membrane, the membrane is washed with a wash buffer (PBS, 0.1% (v/v) Triton X-114, pH 7). The phages bound to the membrane are then eluted with an elution buffer (PBS, continuous gradient: 150 mM to 1 M NaCl, pH 6) (also see FIG. 1). The individual steps of the ion exchange chromatography are analyzed by determining the phages (by means of quantitative ELISA) and endotoxin (by means of an LAL test) concentrations.

Although a depletion of the endotoxins can be achieved with ion exchange chromatography, complete removal is not possible.

### Example 3

Loading the microporous Sartobind® membrane, IDA type 19442, made of regenerated cellulose reinforced with fleece, with Cu²⁺ ions (IDA=imido-diacetic acid).

The regenerated cellulose has been stabilized against enzymatic and chemical degradation by incorporating bifunctional chemical groups. In addition, polymethacrylate chains were grafted on, with the individual monomers each containing an epoxy group, to which chemical imido-diacetic acid groups were coupled. The membrane is 275±27 μm thick and, when lightly buffered aqueous saline solutions in the pH range of 5 to 9 are used, has a flow rate of more than 80 ml/min-bar.

The nominal pore size falls within a range of 3 to 5 μm.

The membrane, which is disposed in a plastic casing, is connected to a 50 ml syringe without a piston and treated and washed, respectively, sequentially with 3 ml deionized water, 4 ml pre-filtered 0.3 M CuCl₂ solution, 3 ml deionized water and 5 ml PBS. The membrane treated in this manner is now ready for use in affinity chromatography.

### Example 4

Affinity chromatographic purification of recombinant phages using a Cu²⁺-containing membrane.

2 ml of an M13/fd phage suspension (in PBS), which contains recombinant phages with His-Tag/g8 fusion proteins on the surface, is applied to the Cu²⁺-containing membrane obtained in accordance with example 3 using a peristaltic pump at a flow rate of 0.25 ml/min. The membrane is then washed three times with 5 ml PBS (W1, W2, W3) at a flow-rate of 0.5 ml/ min. and, subsequently, the recombinant phages bound to the membrane are eluted, in sequence, with 2 ml 20 mM EDTA, 2 ml 40 mM EDTA and 20 ml 80 mM EDTA, in each instance at a flow rate of 0.25 ml/min (E1, E2, E3). The affinity chromatographic purification was analyzed with a dot blot using monoclonal peroxidase-conjugated/anti-polyhistidine antibodies.

As is clearly visible in FIG. 2, the recombinant phages are specifically bonded to the Cu²⁺-containing membrane and eluted by means of EDTA.

The membrane used can be reused by washing it sufficiently with 0.5 m EDTA for removal of the Cu²⁺ ions, washing it with PBS/5% SDS in the reverse direction, and by sufficiently washing it with deionized water to remove the SDS, or it can be stored in a sealed container at 4° C.

### Comparative Example 1

Affinity chromatographic purification of recombinant phages using Ni²⁺ agarose beads.

Commercially available Ni²⁺ agarose beads (Amersham Co.) are loaded, using the manufacturer's instructions, with the recombinant phages described in example 4, incubated overnight and then eluted.

The results show that the purification of recombinant bacteriophages obtained with Ni²⁺ agarose beads (see dot blot in FIG. 3(A)) with the Ni²⁺ agarose beads is significantly worse than the purification, shown in FIG. 3(B), with the Cu²⁺ membrane used in accordance with the invention.

1. Method for purifying and/or isolating high-molecular compounds contained in a solution or a suspension with the capacity for metal chelate formation, comprising the steps:
(a) Application of the solution or suspension onto a metal ions containing membrane, and
(b) affinity chromatographic separation of the high-molecular compounds by binding them to the metal ion containing membrane.

2. Method according to claim 1, wherein the high-molecular compounds have a molecular weight greater than 1×10⁶ Da.

3. Method according to claim 1, wherein the high-molecular compounds are selected from the group consisting of high-molecular proteins, high-molecular protein-like compounds, high-molecular biopolymers, high-molecular lipids, micelles having a high molecular weight and liposomes having a high molecular weight.

4. Method according to claim 1, wherein the metal ions are selected from the group consisting of Cu²⁺, Ni²⁺, Zn²⁺, Co²⁺, Fe³⁺, Mn²⁺ and Ca²⁺ and mixtures thereof.

5. Method according to claim 4, wherein the metal ion is Cu²⁺.

6. Method according to claim 1, wherein the membrane is a matrix material selected from the group consisting of agaroses, modified agaroses, modified dextranes, polystyrenes, polyethers, polyacrylamides, polyamides, cellulose, modified celluloses, such as cross-linked celluloses, nitrocelluloses, cellulose acetates, silicates and poly(meth)acrylates, polytetrafluoroethylene, polyesters, polyvinyl chlorides, polyvinylidene fluoride, polypropylene, polysulfones and polyethersulfones.

7. Method according to claim 1, wherein the metal ions containing membrane has a pore size in the range of 0.01 to 12 μm, preferably in the range of 0.45 to 7 μm, especially preferably in the range of 3 to 5 μm.

8. Method according to claim 3, wherein the high-molecular protein-like compounds are selected from the group consisting of (poly)peptides and derivatives thereof, derivatized proteins, recombinant proteins and (poly)peptides, di-, tri-, tetra- to multimers of peptides, polypeptides or proteins, (multi)-protein complexes, cell organelles, fusion proteins, viruses or parts thereof, recombinant viruses or parts thereof, and recombinant bacteriophages or parts thereof.

9. Method according to one of claim 1, wherein a mixture containing the high-molecular compounds is subjected to ion exchange chromatography to remove impurities prior to step (a).

10. Method according to claim 9, wherein the ion exchange chromatography is performed using an ion exchanger membrane.

11. Method according to claim 10, wherein the ion exchanger membrane comprises a matrix material selected from the group consisting of agaroses, modified agaroses, modified dextranes, polystyrenes, polyethers, polyacrylamides, polyamides, cellulose, modified celluloses, such as cross-linked celluloses, nitrocelluloses, cellulose acetates, silicates and poly(meth)acrylates, polytetrafluoroethylenes, polyesters, polyvinyl chlorides, polyvinylidene fluoride, polypropylenes, polysulfones and polyethersulfones.

12. Method according to claim 10, wherein the ion exchanger membrane has a pore size in the range of 0.01 to 12 μm, preferably in the range of 0.45 to 7 μm, and especially preferably in the range of 3 to 5 μm.

13. Method according to claim 10, wherein the functional groups of the ion exchanger membrane are selected from the group consisting of DEAE, DEA, CM, QA, TMA, S, SP and phosphate groups.

14. Method according to claim 9, wherein the impurities comprise bacterial endotoxins, culture medium components and impurities of culture medium components.

15. Method according to claim 1, wherein, prior to step (a) and/or prior to the ion exchange chromatography according to claim 9, a mixture containing the high-molecular compounds is subjected to filtration using a filtration membrane for the removal of additional impurities.

16. Use of the high-molecular compounds purified and/or isolated in accordance with claim 1 as biologically active components in a pharmaceutical composition, which optionally contains a pharmaceutically acceptable carrier and/or diluent.
